(19) United States
(12) Patent Application Publication
(10) Pub. No.: US 2014/0276315 A1
(43) Pub. Date: Sep. 18, 2014

(54) JOINT SUPPORT APPARATUS AND RELATED METHODS

(71) Applicants: Richard E. Gildersleeve, Escondido, CA (US); William E. Nordt, Charles City, VA (US); Ian Kovacevich, Carlsbad, CA (US)

(72) Inventors: Richard E. Gildersleeve, Escondido, CA (US); William E. Nordt, Charles City, VA (US); Ian Kovacevich, Carlsbad, CA (US)

(21) Appl. No.: 13/842,645

(22) Filed: Mar. 15, 2013

(51) Int. Cl. A61F 5/01

(52) U.S. Cl. CPC A61F 5/0102
USPC 602/27

(57) ABSTRACT

An ankle support includes a flexible, elastically stretchable framework configured to generally surround a wearer's ankle when the support is worn, and an anchor band configured to be secured about a lower portion of a wearer's leg. The framework comprises an integrally molded network of interconnected, elastomeric segments defining a plurality of permanent openings. The framework includes an upper securement portion configured to be adjustably secured to the anchor band via the fastening mechanism of the anchor band. The ankle support is configured to allow a wearer of the ankle support to adjustably tension the ankle support by adjusting an extent of the securement portion of the framework passing through a fastening mechanism of the anchor band.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

The present invention generally relates to apparatus for joint support.

Joint fatigue, pain, and instability are common conditions of active and aging people. Such joint ailments often can be attributed to damage to, and degenerative wear in, the contact surfaces of bone ends meeting at the joint. External support in these areas of the body can help address joint fatigue, pain, and/or instability and, generally, external support in various areas of the body can serve to address many different conditions. One or more aspects of the invention provide such support. Moreover, one or more aspects of the invention even augment motion about joints.

A need exists for improvement in apparatus for joint support. This, and other needs, are addressed by one or more aspects of the present invention.

### SUMMARY OF THE INVENTION

The present invention includes many aspects and features. Moreover, while many aspects and features relate to, and are described in, the context of an ankle support, the present invention is not limited to use only in this context, as will become apparent from the following summaries and detailed descriptions of aspects, features, and one or more embodiments of the present invention.

Accordingly, one aspect of the present invention relates to an ankle support comprising a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn; and a tensioning anchor band configured to be secured about a lower portion of a wearer's leg, the anchor band including a fastening mechanism. The framework includes an upper securement portion configured to be adjustably secured to the anchor band via the fastening mechanism of the anchor band. The ankle support is configured to allow a wearer of the ankle support to adjustably tension the ankle support by adjusting an extent of the securement portion of the framework passing through the fastening mechanism.

In a feature of this aspect, the anchor band comprises hook and loop fasteners.

In a feature of this aspect, the anchor band comprises a ratcheting closure mechanism.

In a feature of this aspect, the ankle support is configured to allow a wearer of the ankle support to adjust lateral or medial support of the ankle support by adjusting an extent of the securement portion of the framework passing through the fastening mechanism.

In a feature of this aspect, the framework is configured to provide arch support when worn.

In a feature of this aspect, the framework is shaped to provide arch support when worn.

In a feature of this aspect, a collective cross-sectional shape of the framework defines a shape generally having a circumference.

In a feature of this aspect, the framework is configured to be capable of lying generally flat when the support is not worn.

In a feature of this aspect, the framework comprises a segment configured to lie over a heel of a wearer when the ankle support is worn.

In a feature of this aspect, the ankle support further comprises a hinge member.

In a feature of this aspect, the ankle support is secured to a sleeve.

In a feature of this aspect, the ankle support forms part of an article of clothing.

Another aspect relates to an ankle support comprising a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn; a tensioning anchor band configured to be secured about a lower portion of a wearer's leg, the anchor band including a fastening mechanism; and a stiffening member. The framework includes an upper securement portion configured to be adjustably secured to the anchor band via the fastening mechanism of the anchor band. The ankle support is configured to allow a wearer of the ankle support to adjustably tension the ankle support by adjusting an extent of the securement portion of the framework passing through the fastening mechanism. The ankle support includes a stiffening member.

In a feature of this aspect, the stiffening member is secured to the framework.

In a feature of this aspect, the stiffening member is at least partially embedded in the framework.

In a feature of this aspect, the stiffening member forms part of the framework.

In a feature of this aspect, the stiffening member is constructed from the same material as the framework.

Another aspect relates to a method comprising equipping a wearer with an ankle support such that an anchor band of the ankle support is secured about a lower portion of the wearer's leg, the anchor band including a fastening mechanism, and a flexible, elastically stretchable framework generally surrounds the wearer's ankle, the framework comprising an integrally molded network of interconnected, elastomeric segments defining a plurality of permanent openings, one of the plurality of permanent openings comprising an alignment opening receiving an ankle joint of the wearer, wherein the framework includes an upper securement portion adjustably secured to the anchor band via the fastening mechanism of the anchor band. The method further includes adjusting a tension level of the framework by adjusting an extent of the securement portion of the framework passing through the fastening mechanism of the anchor band.

In a feature of this aspect, equipping a wearer with an ankle support comprises securing the anchor band about the wearer's leg utilizing hook and loop fasteners of the anchor band.

In a feature of this aspect, equipping a wearer with an ankle support comprises securing the anchor band about the wearer's leg utilizing a ratcheting closure mechanism of the anchor band.

In a feature of this aspect, adjusting a tension level of the framework by adjusting an extent of the securement portion of the framework passing through the fastening mechanism of the anchor band comprises adjusting lateral or medial support of the ankle support.

In a feature of this aspect, the framework is configured to provide arch support when worn.

In a feature of this aspect, the framework is shaped to provide arch support when worn.

In a feature of this aspect, a collective cross-sectional shape of the framework defines a shape generally having a circumference.

In a feature of this aspect, the framework is configured to be capable of lying generally flat when the support is not worn.

In a feature of this aspect, the framework comprises a segment configured to lie over a heel of a wearer when the ankle support is worn.

In a feature of this aspect, the method comprises equipping the wearer with a sleeve worn under the elastically stretchable framework. In at least some implementations, the framework is secured to the sleeve.

Another aspect relates to an ankle support comprising a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn; and an anchor band configured to be secured about a lower portion of a wearer's leg, the anchor band including a fastening mechanism. The framework includes an upper securement portion configured to be adjustably secured to the anchor band via the fastening mechanism of the anchor band. The ankle support is configured to allow a wearer of the ankle support to adjustably tension the ankle support by adjusting an extent of the securement portion of the framework passing through the fastening mechanism. The framework is shaped to provide arch support when worn. The ankle support is configured to allow a wearer of the ankle support to adjust lateral or medial support of the ankle support by adjusting an extent of the securement portion of the framework passing through the fastening mechanism.

In a feature of this aspect, the anchor band comprises hook and loop fasteners.

Another aspect relates to an ankle support comprising a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn.

In a feature of this aspect, the framework is secured to a sleeve shaped and dimensioned to be worn about a user's ankle.

In a feature of this aspect, the framework is secured to footwear.

In a feature of this aspect, the framework is secured to an article of clothing.

Another aspect relates to an ankle support comprising a flexible, elastically stretchable framework configured to generally surround a wearer's elbow when the support is worn, the framework comprising an integrally molded network of interconnected, elastomeric segments defining a plurality of permanent openings, one of the plurality of permanent openings comprising an alignment opening configured to receive an elbow joint of a wearer when the support is worn.

In a feature of this aspect, the framework is secured to a sleeve shaped and dimensioned to be worn about a user's elbow.

In a feature of this aspect, the framework is secured to an article of clothing.

Another aspect relates to a joint support comprising a flexible, elastically stretchable framework configured to generally surround a joint of a wearer when the support is worn, the framework comprising an integrally molded network of interconnected, elastomeric segments defining a plurality of permanent openings, one of the plurality of permanent openings comprising an alignment opening configured to receive the joint of the wearer when the support is worn; and a tensioning anchor band configured to be secured about a portion of the wearer's body proximate the joint, the anchor band including a fastening mechanism. The framework includes an upper securement portion configured to be adjustably secured to the anchor band via the fastening mechanism of the anchor band. The joint support is configured to allow the wearer of the joint support to adjustably tension the joint support by adjusting an extent of the securement portion of the framework passing through the fastening mechanism.

In addition to the aforementioned aspects and features of the present invention, it should be noted that the present invention further encompasses the various possible combinations and subcombinations of such aspects and features. Thus, for example, any aspect may be combined with an aforementioned feature in accordance with the present invention without requiring any other aspect or feature.

### BRIEF DESCRIPTION OF THE DRAWINGS

One or more preferred embodiments of the present invention now will be described in detail with reference to the accompanying drawings, wherein the same elements are referred to with the same reference numerals, and wherein,

FIG. 1 illustrates an exemplary ankle support in accordance with one or more preferred implementations.

### DETAILED DESCRIPTION

As a preliminary matter, it will readily be understood by one having ordinary skill in the relevant art (“Ordinary Artisan”) that the present invention has broad utility and application. As should be understood, any embodiment may incorporate only one or a plurality of the above-disclosed aspects of the invention and may further incorporate only one or a plurality of the above-disclosed features. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, one or more preferred embodiments of the present invention are next described. The following description of one or more preferred embodiments is merely exemplary in nature and is in no way intended to limit the invention, its implementations, or uses.

FIG. 1 illustrates a support 100 for an ankle joint in accordance with one or more preferred implementations. The support 100 includes a flexible, elastically stretchable framework 106 that is configured to extend across a wearer's ankle joint.

The flexible framework 106 comprises an elastomeric material and, specifically, the framework 106 defines a flexible, elastically stretchable web of elastomeric interconnected members. The elastomeric interconnected members mostly comprise generally linear or curved segments. Furthermore, the framework 106, and the interconnected members in particular, preferably include no internal cavities or pockets of either fluid or gas. The interconnected members define a plurality of permanent openings in the web that extend completely through the framework 106. The openings are “permanent” in that they exist regardless of whether the framework 106 actually is disposed in abutment with a wearer's body due to the permanent interconnection of the segments defining the openings. Furthermore, some of these openings are completely bounded by the interconnected members, and the interconnected members defining such an opening constitute a portion of the framework 106 that is stretchable and recoverable about the entire boundary of the opening with the framework 106.

The web includes a larger opening that is configured to receive a portion of an ankle joint therethrough and which serves as an alignment opening 107. Specifically, the alignment opening 107 is defined and bounded completely by interconnected members of the framework 106 and is dimensioned and shaped specifically to receive a joint protuberance of the ankle.

The framework 106 is configured to be stretched and tensioned into abutment with the area of a wearer's body proximate his or her ankle such that the framework 106 conforms to the shape and contour of that area of the body when stretched and tensioned. In one or more preferred implementations the framework 106 has a relaxed state when not stretched and tensioned that generally corresponds to an ankle, while in at least some implementations the framework in its relaxed state may be flatter, or even generally flat.

It will be appreciated by an Ordinary Artisan that, due to the elastically stretchable nature of the framework, the support allows movement at the ankle joint of the body and, in fact, contributes to such flexing. In this regard, because at least portions of the framework 106 are elastically stretchable, flexing of the ankle from a first position to a second position results in the expansion and/or contraction of portions of the framework 106 and storage of potential energy therein that is released as kinetic energy when the ankle is returned to the first position. The support 100 thus is not an immobilizing support but, instead, the support 100 is a potentiating support for the area of the body including the ankle joint insofar as the framework 106 is capable of dynamically biasing a joint toward a particular state of extension or flexion.

In particular, because of the elastically stretchable and recoverable nature of the material of the framework 106, the framework 106 stores potential energy when stretched and tensioned that is released as kinetic energy upon transitioning of the framework 106 back toward a relaxed (or less tensioned) state.

In some preferred implementations, a framework of a support may be configured as disclosed in U.S. Pat. No. 7,691,074 or USPA Pub. No. 2009/0259156, which are both hereby incorporated herein by reference.

The support 100 further includes an anchor band 110 configured to be secured about a lower portion of a wearer's leg, as illustrated in FIG. 1. In one or more preferred implementations, the anchor band 110 is configured to be secured via hook and loop fasteners or via a ratcheting closure mechanism.

In one or more preferred implementations, a securement portion 108 is secured to or forms part of the framework 106. The securement portion 108 is configured for adjustable securement (and tensioning) of the framework 106 to the anchor band 110. In one or more preferred implementations, the anchor band includes a loop or ring 112 configured to allow a portion of the securement portion 108 to be passed through and then secured, for example via a clasp, or alternatively or additionally secured to itself, for example via hook and loop fasteners. Although described as a loop or ring, it will be appreciated that this could also be an opening or any other number of securement or retension mechanisms. In one or more preferred implementations, the securement portion 108 and/or a portion of the framework 106 comprises hook and loop fasteners configured to effect securement of the framework 106 to the anchor band 110. Alternatively, or additionally, in one or more preferred implementations, the anchor band 110 includes a ratchet closure system or clasp configured to effect securement of the framework 106 to the anchor band 110.

In some preferred implementations, a support may utilize a closure system such as those disclosed in U.S. Pat. No. 7,691,074 or USPA Pub. No. 2009/0259156, which are both hereby incorporated herein by reference.

In preferred implementations, the securement portion 108 can be adjustably secured to the anchor band 110 and tensioned at various tension levels, for example by increasing or decreasing the extent of the securement portion 108 disposed through the loop or ring 112 of the anchor band 110. In one or more preferred implementations, a wearer can adjust lateral or medial support of the support 100 by adjusting an extent of the securement portion 108 of the framework passing through the loop or ring 112.

In one or more preferred implementations, the framework 106 is configured to provide arch support to a wearer. For example, in one or more preferred implementations, the framework 106 is shaped and dimensioned, and optionally even reinforced, to provide such arch support. In one or more preferred implementations, foot orthotics are integrated with or form part of a framework or support.

In one or more preferred implementations, the framework is configured for distal migration control. Adjustment of the extent of the securement portion 108 passing through the loop or ring 112 might be utilized for distal migration control. Additionally or alternatively, a separate strap might be utilized which is configured to be disposed proximate a top wearer's foot that is configured for distal migration control. In one or more preferred implementations, one or more members of the framework 106 are shaped and positioned for distal migration control.

In one or more preferred implementations, the framework is configured for calcaneal control. Adjustment of the extent of the securement portion 108 passing through the loop or ring 112 might be utilized for calcaneal control. Additionally or alternatively, a separate strap might be utilized which is configured to be disposed proximate a top wearer's foot that is configured for calcaneal control. In one or more preferred implementations, one or more members of the framework 106 are shaped and positioned for calcaneal control.

In one or more preferred implementations, the framework comprises a single framework shaped to generally encircle a wearer's ankle when worn. In one or more preferred alternative implementations, rather than including only a single framework, a support comprises two frameworks, each representing generally a half of a framework portion of the support. In such preferred implementations, one or more straps or other connection members may be utilized to secure the two frameworks together. In at least some preferred implementations, a support may comprise more than two frameworks. In at least some preferred implementations, one or more frameworks of a support are configured to be capable of lying generally flat when not tensioned in abutment with a wearer's ankle.

In one or more preferred implementations, one or more points or portions of a framework is thicker or stiffer than other points or portions of the framework. In one or more preferred implementations, a stiffening member is utilized to stiffen a framework of a support. Such stiffening member may comprise a different material than the framework, or may be formed of the same material.

In one or more preferred implementations, in use, one or more securement portions are grasped and manually pulled at desired levels of tension, whereby the support 100 is highly adjustable. The resulting tensional forces are applied along the framework 106, whereby the framework 106 is elastically stretched and the surface thereof is shaped to fit the abutted area of the body including the ankle joint. Preferably, the framework 106 of the support 100 does not overlap itself when worn.

In one or more preferred implementations, a sleeve having an open-ended tubular structure is utilized with a support. Such a sleeve preferably extends around and completely encircles an ankle of a wearer when worn. The sleeve is preferably constructed of a soft material. The sleeve itself also may be elastically stretchable. Exemplary materials include synthetic and natural fabrics, monolayer and multi-layered textiles, woven and non-woven planar materials, neoprene bonded to fabric, spandex and elastane, felt, and natural and synthetic chamois. At least a portion of the sleeve is preferably disposed, when worn, between the framework and a portion of a wearer's body proximate the ankle and would thereby serve as a liner for the framework. Exemplary support assemblies including a framework and a sleeve are disclosed in U.S. Pat. No. 7,691,074 or USPA Pub. No. 2009/0259156, which are both hereby incorporated herein by reference. This disclosure is incorporated to illustrate features and functionality which is contemplated for use in combination with supports disclosed herein.

In one or more preferred implementations a framework or support is secured to a sleeve, either at an interior or exterior of the sleeve. In one or more preferred implementations, a framework or support is secured to or forms part of a sock, stocking, pair of pants, shirt, other clothing article, or sleeve. In one or more preferred implementations, a framework or support is secured to or forms part of footwear.

Frameworks as disclosed herein may be elastic, flexible, and/or semi-flexible. In one or more preferred implementations, a framework is constructed from a material configured to change density based on one or more factors, such as, for example, temperature, impact, and/or conformity. In one or more preferred implementations, a framework or support includes a hinge incorporated therein configured to facilitate bending of the framework and/or support.

Although FIG. 1 illustrates a framework, and support, having a first length, frameworks and supports of various lengths may be utilized. In one or more preferred implementations, a framework has a variable length.

In one or more preferred implementations, frameworks as disclosed herein preferably are manufactured in injection molding processes, whereby the framework is integrally formed from elastomeric materials. In some implementations, for example some implementations utilizing a stiffening member, the injection molding processes preferably comprise, for each support, multi-step injection molding, whereby each component can be formed from different elastomeric materials having different elastic stretchability even though the components are integrally constructed.

Additionally, the framework may be made of differing elastomeric materials and/or selected groups of interconnected segments of the framework can be made with varying thickness thereby providing different elastic characteristics and, thereby, providing different resistances to stretching in such areas.

The support of FIG. 1 is an ankle support, but it will be appreciated that disclosure herein with respect to an ankle support is applicable to supports for other joints as well, and such supports are specifically contemplated as within the scope of one or more implementations. For example, in one or more preferred implementations, a support is configured for an elbow joint.

Based on the foregoing description, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention. Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

1. An ankle support comprising:
(a) a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn; and
(b) a tensioning anchor band configured to be secured about a lower portion of a wearer's leg, the anchor band including a fastening mechanism;
(c) wherein the framework includes an upper securement portion configured to be adjustably secured to the anchor band via the fastening mechanism of the anchor band; and
(d) wherein the ankle support is configured to allow a wearer of the ankle support to adjustably tension the ankle support by adjusting an extent of the securement portion of the framework passing through the fastening mechanism.

2. The ankle support of claim 1, wherein the anchor band comprises hook and loop fasteners.

3. The ankle support of claim 1, wherein the anchor band comprises a ratcheting closure mechanism.

4. The ankle support of claim 1, wherein the ankle support is configured to allow a wearer of the ankle support to adjust lateral or medial support of the ankle support by adjusting an extent of the securement portion of the framework passing through the fastening mechanism.

5. The ankle support of claim 1, wherein the framework is configured to provide arch support when worn.

6. The ankle support of claim 1, wherein the framework is shaped to provide arch support when worn.

7. The ankle support of claim 1, wherein a collective cross-sectional shape of the framework defines a shape generally having a circumference.

8. The ankle support of claim 1, wherein the framework is configured to be capable of lying generally flat when the support is not worn.

9. The ankle support of claim 1, wherein the framework comprises a segment configured to lie over a heel of a wearer when the ankle support is worn.

10. The ankle support of claim 1, wherein the ankle support further comprises a hinge member.

11. The ankle support of claim 1, wherein the ankle support is secured to a sleeve.

12. The ankle support of claim 1, wherein the ankle support forms part of an article of clothing.

13. An ankle support comprising:
(a) a flexible, elastically stretchable framework configured to generally surround a wearer's ankle when the support is worn, the framework comprising an integrally molded network of interconnected, elastomeric segments defining a plurality of permanent openings, one of the plurality of permanent openings comprising an alignment opening configured to receive an ankle joint of a wearer when the support is worn;
(b) a tensioning anchor band configured to be secured about a lower portion of a wearer's leg, the anchor band including a fastening mechanism; and
(c) a stiffening member;
(d) wherein the framework includes an upper securement portion configured to be adjustably secured to the anchor band via the fastening mechanism of the anchor band;
(e) wherein the ankle support is configured to allow a wearer of the ankle support to adjustably tension the ankle support by adjusting an extent of the securement portion of the framework passing through the fastening mechanism;
(f) wherein the ankle support includes a stiffening member.

14. The ankle support of claim 13, wherein the stiffening member is secured to the framework.

15. The ankle support of claim 13, wherein the stiffening member is at least partially embedded in the framework.

16. The ankle support of claim 13, wherein the stiffening member forms part of the framework.

17. The ankle support of claim 13, wherein the stiffening member is constructed from the same material as the framework.

18. A method comprising:
(a) equipping a wearer with an ankle support such that
(i) an anchor band of the ankle support is secured about a lower portion of the wearer's leg, the anchor band including a fastening mechanism, and
(ii) a flexible, elastically stretchable framework generally surrounds the wearer's ankle, the framework comprising an integrally molded network of interconnected, elastomeric segments defining a plurality of permanent openings, one of the plurality of permanent openings comprising an alignment opening receiving an ankle joint of the wearer,
(iii) wherein the framework includes an upper securement portion adjustably secured to the anchor band via the fastening mechanism of the anchor band; and
(b) adjusting a tension level of the framework by adjusting an extent of the securement portion of the framework passing through the fastening mechanism of the anchor band.

19. The method of claim 18, wherein equipping a wearer with an ankle support comprises securing the anchor band about the wearer's leg utilizing hook and loop fasteners of the anchor band.

20. The method of claim 18, wherein equipping a wearer with an ankle support comprises securing the anchor band about the wearer's leg utilizing a ratcheting closure mechanism of the anchor band.

21-36. (canceled)
